(19) United States
(12) Patent Application Publication
(10) Pub. No.: US 2024/0091298 A1
(43) Pub. Date: Mar. 21, 2024

(54) A PROCESS FOR PREPARING A PHARMACEUTICAL PREPARATION

(71) Applicant: DR DOZO LABORATORIES, Mohali (IN)

(72) Inventor: Gurdial Singh ARORA, Mohali (IN)

(21) Appl. No.: 18/038,418

(22) PCT Filed: Nov. 23, 2021
(86) PCT No.: PCT/IN2021/051092

§ 371 Date: May 23, 2023

(30) Foreign Application Priority Data
Nov. 24, 2020 (IN) 202011051165

(51) Int. Cl. A61K 36/8998; A61P 31/14

(52) U.S. Cl. CPC A61K 36/8998; A61P 31/14; A61K 2236/331; A61K 2236/51

(57) ABSTRACT

The present invention provides a process for preparing a pharmaceutical preparation which is to be used for treatment of the viral infections, specifically the infections caused by related RNA virus such as Coronavirus (COVID-19) disease. The present invention also provides the method of treating viral infections or the infections caused by related RNA viruses such as COVID-19 disease using Hordeum or Barley or Barley extracts or the pharmaceutical preparation comprising them and the combinations thereof. The present process is simple, economical, bio friendly, and industrially applicable.

### FIELD OF THE INVENTION

The present invention relates to the field of pharmaceutical sciences. Accordingly, the present invention relates to a process for preparing a pharmaceutical preparation which is to be used for treatment of the viral infections, specifically the infections caused by related RNA virus such as Coronavirus (COVID-19) disease. The present invention also provides the method of treating viral infections or the infections caused by related RNA viruses such as COVID-19 disease using Hordeum or Barley or Barley extracts or the pharmaceutical preparation comprising them and the combinations thereof. The present process is simple, economical, bio-friendly, and industrially applicable.

### BACKGROUND OF THE INVENTION

Coronaviruses are a group of related RNA viruses that cause diseases in mammals and birds. In humans and birds, they can cause respiratory tract infections which ranges from mild to lethal illness. Mild illnesses in humans include some cases of the common cold (which is also caused by other viruses, predominantly rhinoviruses), while more lethal varieties can cause SARS, MERS, and COVID-19.

Coronavirus disease (COVID-19) is an infectious disease caused by a newly discovered coronavirus. The coronavirus disease (COVID-19) outbreak was declared a public health emergency by the World Health Organization on Jan. 30, 2020. A majority (67-85%) of critically ill patients who were admitted to the ICU with a confirmed infection of severe acute respiratory syndrome coronavirus 2 (SARS-CoV-2) developed acute respiratory distress syndrome (ARDS). The SARS-CoV-2 pandemic has inspired new interest in understanding the fundamental pathology of acute respiratory distress syndrome (ARDS), which has been associated with severe coronavirus disease 2019 (COVID-19).

It is a brainchild of the inventor whose interest is in studying and converting edible material into active targeted therapies and taking care of critical and chronic diseases. That is how the inventor came across Hordeum and started studying for its anti-oxidant and anti-proliferative properties. After performing further research and pre-clinical studies, the inventor observed its anti-cancer activities that are too significant. Further, it was tested into humans which gives encouraging results in areas where present day therapies are not working. It is unique because it is derived from single product Hordeum and then the biotech process is involved bringing efficacy to the final product.

Hordeum is called barley grain and is very commonly cultivated in various parts of the world. It can be grown in summer as well winter seasons. The fields they are grown in are close to the inventor's premises. The grains were first grinded and pulverized to powder form and then biotechnologically processed. First use of Hordeum was done in March 2000 to study its anti-oxidant properties. Raw material was tested for pesticides content and no contents of the pesticides were found.

Earlier pharmaceutical preparations were having the drawback that the preparation was having the acidic pH.

The inventor in the present invention has addressed the problems and developed a novel and inventive process of preparation of pharmaceutical composition. The preparation after lyophilisation was having acidic pH and it is further treated and neutralised by adding basic solvent. With such modifications, the biological activity of pharmaceutical preparation is improved, and cell morphology is retained.

The pharmaceutical preparation has been studied in Ln-Cap cell line which is androgen sensitive Human Prostate cancer cell line. 100% inhibition was achieved at 2% V/V. This study was conducted at Harvard university.

The pharmaceutical preparation of present invention has shown anti-viral activity, preferably against the RNA related virus, more preferably against the Coronavirus (COVID-19). Accordingly, the present invention provides a method of preparing the pharmaceutical preparation that can be utilized in the treatment of viral infections, specifically, RNA virus infections, more specifically, COVID-19 infections. Also provided is the method of treating viral infections using Hordeum or Barley or Barley extracts or the pharmaceutical preparation comprising them and their uses thereof.

### OBJECTS OF THE PRESENT INVENTION

The main object of the present invention is to provide a process of preparing a pharmaceutical preparation comprising Hordeum or Barley or Barley extracts.

Another object of the present invention is to provide a process of preparing a pharmaceutical preparation which have applications in the treatment of viral infections, or the infections caused by related RNA viruses such as COVID-19 disease.

Another object of the present invention is to provide a method of treating viral infections or the infections caused by related RNA viruses such as COVID-19 disease using Hordeum or Barley or Barley extracts or the pharmaceutical preparation comprising them.

Another object of the present invention is to provide a process which is simple, economical, bio-friendly, and industrially applicable.

### SUMMARY OF THE INVENTION

The present invention provides a process for preparing a pharmaceutical preparation which is to be used for treatment of the viral infections, specifically the infections caused by related RNA virus such as Coronavirus (COVID-19) disease. The present invention also provides the method of treating viral infections or the infections caused by related RNA viruses such as COVID-19 disease using Hordeum or Barley or Barley extracts or the pharmaceutical preparation comprising them and the combinations thereof. The present process is simple, economical; bio-friendly, and industrially applicable.

### DETAILED DESCRIPTION OF THE INVENTION

It is the novel idea by the inventor and used for said diseases. Accordingly, the present invention relates to a process for preparing a pharmaceutical preparation which is to be used for treatment of the viral infections, specifically the infections caused by related RNA virus such as Coronavirus (COVID-19) disease.

The process comprises of the steps of mixing barley flour (obtained by grinding Barley grains) with water and vigorously stirred. The solution is then kept at a temperature 27±3° C. and is kept at the temperature for 16 hours, then subjected to distillation at 80-140° C. The distillate so obtained is covered properly and kept at temperature 10±2° C. for one hour. Furthermore, after the product is distilled, it is Lyophilised and diluted to different concentration to have enhanced activity.

Further, the pharmaceutical preparation after lyophilisation was having pH 3.5 which is then neutralised by adding NaOH (Sodium Hydroxide) to the preparation to bring the pH level to 7.2. With this step, the biological activity was enhanced, and cell morphology was retained.

During anti-viral cell culture studies the big problem surfaced during studies of treated cells found that cells morphology is changed. As earlier preparations were strongly acidic with pH around 3.5, it was neutralized with 1N NaOH to make it alkaline and bringing the pH to around 7.4.

With the preparation neutralized was tested with viral cell lines. The Morphology of treated cells were retained. The activity of the pharmaceutical preparation was also increased as observed in experimental results even at 75× times dilutions gave same results that of 50× acidic preparation. It gave the inventors a clear indication that pharmaceutical preparation in alkaline nature is more effective and retain cell morphology while neutralizing the viruses.

According to a preferred embodiment of the present invention, the Barley flour is mixed with distilled water, more preferably double distilled water.

The solution thus obtained is the pharmaceutical preparation, desired to be prepared. Satcon is the name of the final product obtained from this pharmaceutical preparation. Satcon is a C-JNK Kinase inhibitor pharmaceutical preparation so proposed Mechanism of Action for the pharmaceutical preparation observed is that it protects epithelial cells of respiratory tract as it inhibits C-JNK pathway which is activated in cells infected by coronavirus infectious bronchitis virus (IBV). C-JNK induces proinflammatory cytokines such as TNFa, IFNs, IL1, IL2 and IL6, so inhibition of this kinase could lead to anti-inflammatory effects and JNK induces activation of transcription factor such as NF-KB p65, C-Jun which plays a pivotal role in regulating immune response to viral infections (Nature Journal, Article 3215 by Sing Fung and Ding Xiang Liu, 2017). This study was conducted by Fluofarma in April 2012 to see the effects of Satcon on different pathways.

According to another preferred embodiment of the invention, the solution of Barley flour after being kept at 27±3° C. for 16±2 hours, subjected to distillation at a temperature 80 to 140° C.

According to another preferred embodiment of the present invention, Barley flour and water are mixed in 1:2 ratio respectively. Various means were tried to obtain solids after 2n d step with drying by known methods of drying, but optimum results were obtained through distillation route.

Furthermore, after the product is distilled, it is Lyophilised and diluted to different concentration to have enhanced activity.

Another embodiment of the present invention provides a method of treating viral infections or the infections caused by related RNA viruses such as COVID-19 disease using Hordeum or Barley or Barley extracts and the combinations thereof.

Another embodiment of the present invention provides a use of Hordeum or Barley or Barley extracts and the combinations thereof and their pharmaceutical preparation in the treatment of viral infections or the infections caused by related RNA viruses such as COVID-19 disease.

Another embodiment of the present invention provides a method of treating viral infections or the infections caused by related RNA viruses such as COVID-19 disease using the pharmaceutical preparation which comprises Hordeum or Barley or Barley extracts and the combinations thereof.

Another embodiment of the present invention provides a process of preparing the pharmaceutical preparation wherein the process is simple, economical, bio-friendly, and industrially applicable.

A preferred embodiment of the present invention provides a pharmaceutical preparation of Hordeum or Barley or Barley extracts and the combinations thereof which is be used in the treatment of the Coronavirus (COVID-19) disease.

The terms “treatment,” “treat,” “treating,” and the like, are meant to include slowing or reversing the progression of a disease or disorder. These terms also include alleviating, ameliorating, attenuating, eliminating, or reducing one or more symptoms of a disease or disorder or condition, even if the disease or disorder or condition is not actually eliminated and even if progression of the disease or disorder or condition is not itself slowed or reversed.

As explained above, the pharmaceutical preparation of the invention is useful in treating or preventing viral infections or the infections caused by related RNA viruses such as COVID-19 disease. The present invention therefore provides pharmaceutical preparation for use in treating or preventing viral infections or the infections caused by related RNA viruses such as COVID-19 disease. Also provided is a method for treating a patient suffering from or susceptible to viral infections or the infections caused by related RNA viruses such as COVID-19 disease, which method comprises administering to said patient an effective amount of a pharmaceutical preparation. Further provided is the use of a pharmaceutical preparation, in the manufacture of a medicament for use in treating or preventing viral infections or the infections caused by related RNA viruses such as COVID-19 disease.

The main embodiment of the present invention provides a process of preparing a pharmaceutical preparation, wherein the process comprising the steps of:
a) Mixing barley flour with water and stirred vigorously to form a solution.
b) Keeping the solution of step (a) at an appropriate temperature range of 20 to 40° C. for adequate time ranging 10 to 20 hours to form another solution.
c) The solution of step (b) is subjected to distillation at 50 to 200° C. to form a distillate.
d) The distillate formed in step (c) is covered and keeping at temperature of 10±5° C. for time duration of 1 to 3 hour to form a crude product.
e) The crude product is further distilled, Lyophilized, and diluted to different concentrations to obtain a final pharmaceutical preparation.
f) The pharmaceutical preparation of step (e) is treated with basic solvent to make the pH to alkaline levels.

In another embodiment of the present invention, barley flour is obtained by grinding Barley grains.

In another embodiment of the present invention, the water is distilled water.

In another embodiment of the present invention, the distilled water is more preferably double distilled water.

In another embodiment of the present invention, the Barley flour and water are mixed in a ratio of 1:1 to 1:4, preferably, 1:2.

In another embodiment of the present invention, the appropriate temperature of step (b) is in the range of 20 to 30° C.

In another embodiment of the present invention, the appropriate temperature of step (b) is preferably 27±3° C.

In another embodiment of the present invention, the adequate time of step (b) ranges from 12 to 17 hours.

In another embodiment of the present invention, the adequate time of step (b) is 16 hours.

In another embodiment of the present invention, the distillation is carried out at temperature 60 to 150, more preferably 80-140° C.

In another embodiment of the present invention, the temperature of step (d) is 10±4° C., more preferably 10±2° C.

In another embodiment of the present invention, the time duration of step (d) is time duration of 1 to 2 hour, more preferably, one hour.

In another embodiment of the present invention, the basic solvent of step (f) is 1N NaOH.

In another embodiment of the present invention, the pH of step (f) is in the range of 7.2 to 8.0.

In another embodiment of the present invention, the pH of step (f) is preferably, 7.5, more preferably 7.4.

In yet another embodiment of the present invention, the pharmaceutical preparation has application in the treatment of viral infections, or the infections caused by related RNA viruses such as COVID-19 disease.

Another embodiment of the present invention provides a method of treating viral infections or the infections caused by related RNA viruses such as COVID-19 disease using Hordeum or Barley or Barley extracts or the pharmaceutical preparation as prepared in process of present invention.

Another embodiment of the present invention provides use of the pharmaceutical preparation as prepared in process of present invention for the treatment of viral infections or the infections caused by related RNA viruses such as COVID-19 disease.

Yet another embodiment of the present invention provides a process of preparing a pharmaceutical preparation, wherein the process comprising the steps of:
a) Mixing barley flour with double distilled water and stirred vigorously to form a solution.
b) Keeping the solution of step (a) at an appropriate temperature range of 27±3° C. for 16 hours to form another solution.
c) The solution of step (b) is subjected to distillation at 80-140° C. to form a distillate.
d) The distillate formed in step (c) is covered and keeping at temperature of 10±2° C. for one hour to form a crude product.
e) The crude product is further distilled, lyophilized, and diluted to different concentrations to obtain a final pharmaceutical preparation.
f) The pharmaceutical preparation of step (e) is treated with 1N NaOH to make the pH 7.4.

In yet another embodiment of the present invention, the pharmaceutical preparation has application in the treatment of viral infections, or the infections caused by related RNA viruses such as COVID-19 disease.

Another embodiment of the present invention provides a method of treating viral infections or the infections caused by related RNA viruses such as COVID-19 disease using Hordeum or Barley or Barley extracts or the pharmaceutical preparation as prepared in process of present invention.

Another embodiment of the present invention provides use of the pharmaceutical preparation as prepared in process of present invention for the treatment of viral infections or the infections caused by related RNA viruses such as COVID-19 disease.

Yet another embodiment of the present invention provides a process of preparing a pharmaceutical preparation, wherein the process comprising the steps of:
a) Mixing barley flour with double distilled water and stirred vigorously to form a solution.
b) Keeping the solution of step (a) at an appropriate temperature range of 27±3° C. for 16 hours to form another solution.
c) The solution of step (b) is subjected to distillation at 80-140° C. to form a distillate.
d) The distillate formed in step (c) is covered and keeping at temperature of 10±2° C. for one hour to form a crude product.
e) The crude product is further distilled, lyophilized, and diluted to different concentrations to obtain a final pharmaceutical preparation.

In yet another embodiment of the present invention, the pharmaceutical preparation has application in the treatment of viral infections, or the infections caused by related RNA viruses such as COVID-19 disease.

Another embodiment of the present invention provides a method of treating viral infections or the infections caused by related RNA viruses such as COVID-19 disease using Hordeum or Barley or Barley extracts or the pharmaceutical preparation as prepared in process of present invention.

Another embodiment of the present invention provides use of the pharmaceutical preparation as prepared in process of present invention for the treatment of viral infections or the infections caused by related RNA viruses such as COVID-19 disease.

### Examples

The scope of the present invention is illustrated by the following examples as disclosed herein which are not meant to restrict the scope of the invention in any manner whatsoever.

Process of Preparing a Pharmaceutical Preparations:

Preparation-I

The steps comprising of:
Preparing a solution by mixing barley flour with water.
Keeping the solution formed at a temperature range of 20 to 40° C. for 10 to 20 hours to form another solution. This another solution is subjected to distillation at 50 to 200° C. to form a distillate.
Covering the distillate and keeping it at temperature of 10±5° C. for time duration of 1 to 3 hour to form a crude product. Crude product is further distilled, lyophilized, and diluted to different concentrations to obtain a final pharmaceutical preparation.
The pharmaceutical preparation after lyophilization is having pH 3.5 (acidic pH) which is neutralized by adding 1N basic solvent i.e., NaOH (Sodium Hydroxide) to the preparation to bring the pH level in the range of 7.0 to 7.5, preferably 7.4.

Preparation-II

The process comprising the steps of:
Mixing barley flour with double distilled water and stirred vigorously to form a solution.
Keeping the solution of step (a) at an appropriate temperature range of 27±3° C. for 16 hours to form another solution.
The solution of step (b) is subjected to distillation at 80-140° C. to form a distillate.
The distillate formed in step (c) is covered and keeping at temperature of 10±2° C. for one hour to form a crude product.
The crude product is further distilled, lyophilized, and diluted to different concentrations to obtain a final pharmaceutical preparation.

Pre-Clinical Studies:

Anti-viral screening of the pharmaceutical preparation was done at regional centre of biotechnology. The pharmaceutical preparation was tested in 1×10e4 VeroE6 cells and the cells were infected with SARS-CoV2 at a MOI of 0.01. 24 and 48 hours later, viral RNA was extracted from 100 μl culture supernatant and subjected to qRTPCR (in duplicates) where Ct values for N and E gene sequence were determined. Inhibition of virus replication is determined based on the fold change in the Ct value in TS-treated cells compared to the control. Our pharmaceutical preparation gave more than 50 percent inhibition at very low dose of 1 ul that too ten times diluted solution in DMSO.

Toxicity Study Assay Description:

Study was performed as per the OECD TG 425 (Acute Oral Toxicity—Up-and-Down-Procedure (UDP) at UNIVERSITY INSTITUTE OF PHARMACEUTICAL SCIENCES, PANJAB UNIVERSITY CHANDIGARH-160014, INDIA.

The test consists of a single ordered dose progression in which Wistar rats are dosed, one at a time, at a minimum of 48-hour intervals. The first wistar rat receives a dose a step below the level of the best estimate of the LD50. If the rat survives, the dose for the next rat is increased by [a factor of] 3.2 times the original dose; if it dies, the dose for the next rat is decreased by a similar dose progression. (3.2 is the default factor corresponding to a dose progression of one half log unit. Each rat should be observed carefully for up to 48 hours before making a decision on whether and how much to dose the next rat. That decision is based on the 48-hour survival pattern of all the wistar rats up to that time. A combination of stopping criteria is used to keep the number of rats low while adjusting the dosing pattern to reduce the effect of a poor starting value or low slope. Dosing is stopped when one of these criteria is satisfied, at which time an estimate of the LD50 and a confidence interval are calculated for the test based on the status of all the rats at termination.

No mortality was observed in any of the rats and at all tested doses. Histopathological Findings: Observations were made on rats having pharmaceutical preparation administered the assumed/default LD50 for gross pathological changes. NO evident changes for any severe toxicity were observed. Non-clinical data is there for this pharmaceutical preparation and has proven it to be very safe and non-toxic even at high doses of 2000 mg/kg.

Chemistry, manufacturing, and controls data (CMC)
The raw material is Agro based which is sourced from various parts of the country.
The Final product is obtained through Novelty of Reactions (Biotech).
HPGLC fingerprinting of the raw material ensures consistent quality.
Final product batches monitored by HPGLC.

Certificate of Analysis:

Clinical Pharmacology:

The pharmaceutical preparation is administered orally. It is well absorbed. As observed from Human data, it does not contradict nor create negativity with Food and other drugs. All blood parameters remain normal and patients who were having medications for Thyroid, Heart, Diabetes, neuro-defects, rheumatic diseases, Painkillers like (Ultracet, Tramadol), liver diseases were continuing taking this pharmaceutical preparation along with their prescribed medication. Usually, no adverse effects were reported. If patient is having constipation, can have laxatives and anti-acids also for Gastric relief. It crosses the blood-brain barrier. The pharmacokinetics is not altered in patients with renal or liver diseases. The above all information is based on clinical study performed in patients.

Safety

The pharmaceutical preparation has been administered to 12 patients. Out of 12, seven have co-morbities for like high blood sugar levels and high blood pressure. They were given medication for 6-7 days and after completion of course, they turned out negative.

SARS-CoV2 Antiviral Testing

Study 1:

The purpose of the study is to explore and evaluate the EC 50 value of pharmaceutical preparation against SARS-CoV-2 virus.

Methodology

For EC₅₀ estimation. the Vera cells were infected with SARS-CoV-2. After infection. the cells were treated with different concentrations of pharmaceutical preparation (2 μl/ml, 4 μl/ml, 8 μl/ml, 12 μl/ml) respectively followed by collection of respective supernatants at 22 hpi.

Result

| | | | Mean Ct | Copy | % |
|---|---|---|---|---|---|
| S. No. | Compound | Concentration | value | number/ML | Reduction |
| 1. | Infected only | | 23.800 | 124725.7238 | |
| 2. | Pharmaceutical | 2 μl/ml | 24.600 | 73712.51542 | 40.90031056 |
| | preparation | | | | |
| 3. | Pharmaceutical | 4 μl/ml | 25.600 | 38196.42615 | 69.37566287 |
| | preparation | | | | |
| 4. | Pharmaceutical | 8 μl/ml | 28.700 | 4976.401073 | 96.01012452 |
| | preparation | | | | |
| 5. | Pharmaceutical | 12 μl/ml | 29.800 | 2414.599038 | 98.06407294 |
| | preparation | | | | |

The IC₅₀ value of pharmaceutical preparation against SARS CoV-2 is 2.536 μM.

Study-2:

The purpose of the study is to evaluate the antiviral activity of pharmaceutical preparation against SARS-CoV-2 virus.

Study Design
a) To check the cytotoxicity using cultured cells via MTT assay.
b) To evaluate the antiviral effect using highest concentration of sample with lower cytotoxicity.

Methodology

The MTT assay was carried out as per the protocol and the highest permissible non cytotoxic concentration was taken forward for assessing their anti-SARS-activity.

Cytotoxicity Assay: The cytotoxicity assay will be performed as per the kit used for the assay. Briefly, Vero E6 cells will be seeded in the 96 well plate at 80% confluency and treated with various concentrations of the test compounds or extracts. For toxicity determination minimum of 3 concentrations will be used. 48 h post-treatment the MTT assay will be performed according to the manufacturer's instructions. Each concentration will be assayed in triplicates and the percentage cell viability will be calculated with respect to vehicle control.

Antiviral Activity Assay: The assay will be performed as per the protocol routinely followed for determining antiviral activity against SARS-CoV2. Known inhibitors of SAR-CoV2 will be used as positive control in the assay. Vero E6 cells seeded in 96 well plates at 80% confluency will be infected with SARS-CoV-2 isolate at an MOI of 0.1 for 2 h. Subsequently the inoculum will be aspirated and fresh media containing different concentrations of the test compounds/extract will be added to the cells. Each concentration will be assayed in triplicates. Compounds showing more the 50% anti-SARS-CoV2 activity will be considered for IC50 determination. IC50 will be determined using a minimum of 7 different concentrations. 24 h post-infection the supernatant and cells will be subjected to viral RNA isolation followed by qRT-PCR for determining the SARS-CoV-2 viral load in the cells (cell associated) and culture supernatants (released virus particles). qRT-PCR will be performed using primers specific for the viral spike, nucleocapsid and ORF1a for both released and cell-associated virus. Percentage reduction of viral loads in cells and culture supernatants will be plotted in comparison to vehicle treated controls.

| | | Ct | Copy | |
|---|---|---|---|---|
| Sample | Concentration | value | number/ml | % Inhibition |
| Infection | | 25.730 | 35057.96768 | |
| (0.1 MOI) | | | | |
| Pharmaceutical | 12.5 μl/ml | 30.440 | 1584.997638 | 95% |
| preparation | | | | |
| (SACOV ILS- | | | | |
| BV-2021-020) | | | | |

The pharmaceutical preparation gave 95% inhibition which indicates that it is a very strong inhibitor of sars-cov2 virus.

CoV2-19 Efficacy In Vitro:

For infection, the Vera cells were infected with SARS-CoV2-19 as per the protocol. After infection. the cells were treated with 12.5 μl/m1 of pharmaceutical preparation, followed by collection of respective supernatants at 22 hpi.

Result
MTT Assay: Maximum non-toxic dose of pharmaceutical preparation was obtained at 12.5 μl/m1 (88% viability)
Antiviral Assay: Mean Ct value obtained by quantitative real time PCR analysis was found to be 30.440 (approximately 95% reduction as compared to control).
Infected only (control): Mean Ct value is 25.730.

Covid-19 Pseudovirus Assay

Reagents & Materials

Cells Expressing ACE2 Receptor
A549

Cell Culturing
DMEM complete growth medium with 10% FBS and 2% penicillin-streptomycin
Trypsin
Cell counter hemocytometer
U shape 96-well cell culture plate
37° C. incubator with 5% CO2
Microscope

Pipettes, Syringes, & Tubes
Sterile serological pipettes (5 ml, 10 ml, and 25 ml)
Micropipettes
Multichannel micropipettes
Micropipette tips (10 μl, 200 μl, and 1000 μl)
Plastic syringe (1 ml)
Polypropylene sterile conical tubes (15 ml and 50 ml)
96-well cell culture plate (white plate with flat, clear bottom)

Pseudovirus
Supernatant containing the pseudoviruses rVSV-ΔG-pCAGGS/Spike-luciferase

Detection
Firefly luciferase substrate
Luminometer (BioTek synergy HT)

Other
Biological safety cabinet
Sterile reservoirs
Water Bath
Timer
70% Ethanol
Millex GV Filter Unit (Hydrophilic Durapore membrane)
Pharmaceutical Preparation sample(s)

Procedure

Day 0: Culture Cells-of-Interest

Day 1: Seed Cells-of-Interest

20,000 cells per well are seeded in a 96-well white cell culture plate with flat, clear bottom for 24 hours at 37° C./5% CO2.

Day 2: Cell Infection with Generated rVSV Pseudoviruses

Preparations:

Incubation with pseudovirus first: 50-, 75-, and 250-fold final dilutions. The 50-fold dilution are adjusted to pH 7.4.

Incubation with cells first: 50-, 75-, 100-, and 250-fold final dilutions. The 50- and 100-fold dilutions are adjusted to pH 7.4.

Protocol

1.) Vortexed and spun down the sample vial.

2.) Incubated samples with the pseudovirus first.

a. 50-fold dilution of sample (pH 7.4): Neutralized with 1N NaOH to reach pH 7.4 and filtered with 0.22 μM (Millex: Cat #: SLGV004SL). Mixed 2.4 μL, stock solution with 57.6 μL, DMEM. Added the mixed sample to the 96-well plate (samples now are 25-fold diluted). Added 60 μL, pseudovirus to the plate (samples now are 50-fold diluted).

b. 75-fold dilution of sample: Mixed 1.6 μL, stock solution with 58.4 μL, DMEM. Added the mixed sample to the 96-well plate (samples now are 37.5-fold diluted). Added 60 μL, pseudovirus to the plate (samples now are 75-fold diluted).

c. 250-fold dilution of sample: Mixed 0.48 μL, stock solution with 59.52 μL, DMEM. Added the mixed sample to the 96-well plate (samples now are 125-fold diluted). Added 60 μL, pseudovirus to the plate (samples now are 250-fold diluted).

d. Incubated the sample/pseudovirus mixture for 1 hour at 37° C./5% CO2.

e. Removed media from cells prepared on Day 1. Added 100 μL, of diluted sample/pseudovirus mixture to the 96-well plate seeded with cells.

3.) Incubated samples with cells first.

a. 50-fold dilution of sample (pH 7.4): Neutralized the sample with 1N NaOH to reach pH 7.4. Removed media from cells prepared on Day 1. Mixed 1.2 μL, stock solution with 58.8 μL, DMEM. Added 60 μL, mixed sample to each well of cells in the 96-well plate (samples now are 50-fold diluted).

b. 100-fold dilution of sample (pH 7.4): Neutralized the sample with 1N NaOH to reach pH 7.4. Mixed 2.4 μL, stock solution with 57.6 μL, DMEM. Removed media from cells prepared on Day 1. Added 60 μL, mixed sample to each well of cells in the 96-well plate (samples now are 100-fold diluted).

c. 75-fold dilution of sample: Mixed 0.8 μL, stock solution with 59.2 μL, DMEM. Removed media from cells prepared on Day 1. Added 60 μL mixed sample to each well of cells in the 96-well plate (samples now are 75-fold diluted).

d. 250-fold dilution of sample: Mixed 0.24 μL stock solution with 59.76 μL, DMEM. Removed media from cells prepared on Day 1. Added 60 μL mixed sample to each well of cells in the 96-well plate (samples now are 250-fold diluted).

e. Incubated the sample/cell mixture for 1 hour at 37° C./5% CO2.

f. Added 60 μl pseudovirus to the wells.

4.) Incubated the plate for 1.5 hours at 37° C./5% CO2.

5.) Added 100 μl pre-warmed DMEM complete growth medium.

6.) Incubated the plate for 24 hours at 37° C./5% CO2.

Sample Preparation

Negative Control (Mock Infection):

No pseudovirus was added to account for background noise.

Day 3: Luciferase Assay
Removed 200 μL of supernatants from each well.)
Added 50 μL of luciferase substrate to each well. Shaked the plate for 3 minutes at 300-600 rpm to lyse cells and equilibrate samples.
Measured luminescence in a luminometer with an integration time of 0.5-1 second and recorded the results.

Day 4: Data Analysis

1.) Background subtraction: The average optical density (OD) reading of the “mock infection” negative control replicates is subtracted from all samples' OD readings.

2.) Inhibition rate (%):

Inhibition rate=[(“No Sample” positive control OD−sample OD)/“No Sample” positive control OD]×100%

Results:

The preparation is inhibiting pseudoviral entry for both pseudoviruses tested. There was observed a nice stepwise decrease in viral entry neutralizing activity with higher dilutions, which demonstrates that the neutralizing effect is real. This effect was observed with Spike and the non-Spike glycoprotein, especially when the preparation was mixed with pseudovirus first. Two pseudoviruses, Spike and non-Spike glycoprotein were tested. The sample inhibited both viruses, so does not appear to be specific to Spike. Furthermore, the anti-viral properties are more effective when the sample is incubated with the pseudovirus first.

Inhibition Rate (%): 99.77%

| Sample concentration | Inhibition |
|---|---|
| (dilution) | Rate (%) |
| Pesudovirus first (1:75) | 99.77681 |
| Pesudovirus first (1:250) | 70.81869 |
| Pesudovirus first (1:500) | 41.6263 |
| Pesudovirus first (1:1000) | 24.28689 |
| Cells first (1:75) | 27.63637 |
| Cells first (1:250) | 15.63776 |
| Cells first (1:500) | 12.64919 |
| Cells first (1:1000) | −0.70757 |

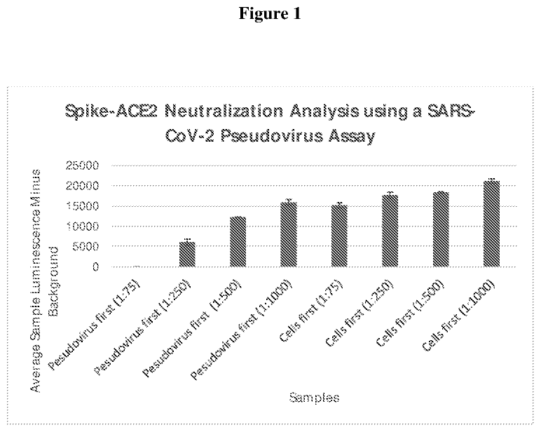

The results of the studies are mentioned in FIG. 1:

The anti-viral properties are more effective when the sample is incubated with the pseudovirus first. At 1:75 dilution compound gave 99.77% inhibition which proves it to be strong inhibitor of sars-cov2 pseudovirus.

The claims:

1. A process of preparing a pharmaceutical preparation, wherein the process comprising the steps of:
a) Mixing barley flour with water and stirred vigorously to form a solution.
b) Keeping the solution of step (a) at an appropriate temperature range of 20 to 40° C. for adequate time ranging 10 to 20 hours to form another solution.
c) The solution of step (b) is subjected to distillation at 50 to 200° C. to form a distillate.
d) The distillate formed in step (c) is covered and keeping at temperature of 10±5° C. for time duration of 1 to 3 hour to form a crude product.
e) The crude product is further distilled, Lyophilized, and diluted to different concentrations to obtain a final pharmaceutical preparation.
f) The pharmaceutical preparation of step (e) is treated with basic solvent to make the pH to alkaline levels.

2. The process as claimed in claim 1, wherein barley flour is obtained by grinding Barley grains.

3. The process as claimed in claim 1, wherein the water is distilled water.

4. The process as claimed in claim 2, wherein the distilled water is more preferably double distilled water.

5. The process as claimed in claim 1, wherein Barley flour and water are mixed in a ratio of 1:1 to 1:4, preferably, 1:2.

6. The process as claimed in claim 1, wherein appropriate temperature of step (b) is in the range of 20 to 30° C.

7. The process as claimed in claim 1, wherein appropriate temperature of step (b) is preferably 27±3° C.

8. The process as claimed in claim 1, wherein the adequate time of step (b) ranges from 12 to 17 hours.

9. The process as claimed in claim 1, wherein the adequate time of step (b) is 16 hours.

10. The process as claimed in claim 1, where distillation is carried out at temperature 60 to 150, more preferably 80-140° C.

11. The process as claimed in claim 1, wherein the temperature of step (d) is 10±4° C., more preferably 10±2° C.

12. The process as claimed in claim 1, wherein the time duration of step (d) is time duration of 1 to 2 hour, more preferably, one hour.

13. The process as claimed in claim 1, where basic solvent of step (f) is 1N NaOH.

14. The process as claimed in claim 1, wherein the pH of step (f) is in the range of 7.2 to 8.0.

15. The process as claimed in claim 14, wherein the pH is preferably, 7.5, more preferably 7.4.

16. The process as claimed in claim 1, wherein the pharmaceutical preparation has application in the treatment of viral infections, or the infections caused by related RNA viruses such as COVID-19 disease.

17. A method of treating viral infections or the infections caused by related RNA viruses such as COVID-19 disease using Hordeum or Barley or Barley extracts or the pharmaceutical preparation as prepared in process of claim 1.

18. Use of the pharmaceutical preparation as prepared in process of claim 1 for the treatment of viral infections or the infections caused by related RNA viruses such as COVID-19 disease.

19. A process of preparing a pharmaceutical preparation, wherein the process comprising the steps of:
a) Mixing barley flour with double distilled water and stirred vigorously to form a solution.
b) Keeping the solution of step (a) at an appropriate temperature range of 27±3° C. for 16 hours to form another solution.
c) The solution of step (b) is subjected to distillation at 80-140° C. to form a distillate.
d) The distillate formed in step (c) is covered and keeping at temperature of 10±2° C. for one hour to form a crude product.
e) The crude product is further distilled, lyophilized, and diluted to different concentrations to obtain a final pharmaceutical preparation.
f) The pharmaceutical preparation of step (e) is treated with 1N NaOH to make the pH 7.4.

20. The process as claimed in claim 19, wherein the pharmaceutical preparation has application in the treatment of viral infections, or the infections caused by related RNA viruses such as COVID-19 disease.

21. A method of treating viral infections or the infections caused by related RNA viruses such as COVID-19 disease using Hordeum or Barley or Barley extracts or the pharmaceutical preparation as prepared in the process of claim 19.

22. Use of the pharmaceutical preparation as prepared in the process of claim 19 for the treatment of viral infections or the infections caused by related RNA viruses such as COVID-19 disease.

23. A process of preparing a pharmaceutical preparation, wherein the process comprising the steps of:
a) Mixing barley flour with double distilled water and stirred vigorously to form a solution.
b) Keeping the solution of step (a) at an appropriate temperature range of 27±3° C. for 16 hours to form another solution.
c) The solution of step (b) is subjected to distillation at 80-140° C. to form a distillate.
d) The distillate formed in step (c) is covered and keeping at temperature of 10±2° C. for one hour to form a crude product.
e) The crude product is further distilled, lyophilized, and diluted to different concentrations to obtain a final pharmaceutical preparation.

24. The process as claimed in claim 23, wherein the pharmaceutical preparation has application in the treatment of viral infections, or the infections caused by related RNA viruses such as COVID-19 disease.

25. A method of treating viral infections or the infections caused by related RNA viruses such as COVID-19 disease using Hordeum or Barley or Barley extracts or the pharmaceutical preparation as prepared in the process of claim 23.

26. Use of the pharmaceutical preparation as prepared in the process of claim 23 for the treatment of viral infections or the infections caused by related RNA viruses such as COVID-19 disease.
